(19) United States
(12) Patent Application Publication
(10) Pub. No.: US 2024/0049981 A1
(43) Pub. Date: Feb. 15, 2024

(54) SYSTEMS AND METHODS FOR ESTIMATION OF FORCED VITAL CAPACITY USING SPEECH ACOUSTICS

(71) Applicants: Arizona Board of Regents on behalf of Arizona State University, Scottsdale, AZ (US); Aural Analytics, Inc., Scottsdale, AZ (US)

(72) Inventors: Visar Berisha, Tempe, AZ (US); Julie Liss, Scottsdale, AZ (US); Shira Hahn, Tempe, AZ (US); Gabriela Stegmann, Mesa, AZ (US); Jeremy Shefner, Scottsdale, AZ (US)

(21) Appl. No.: 18/266,563

(22) PCT Filed: Dec. 9, 2021
(86) PCT No.: PCT/US21/62660

§ 371 Date: Jun. 9, 2023

Related U.S. Application Data
(60) Provisional application No. 63/123,265, filed on Dec. 9, 2020.

(51) Int. Cl. A61B 5/08; G10L 25/66; G10L 15/22; G10L 15/02; A61B 5/00; G16H 10/20; G16H 10/60; G16H 20/30

(52) U.S. Cl. CPC A61B 5/08; G10L 25/66; G10L 15/22; G10L 15/02; A61B 5/4803; A61B 5/7267; A61B 5/6898; G16H 10/20; G16H 10/60; G16H 20/30; A61B 2562/0204

(57) ABSTRACT

Described are platforms, systems, media, and methods for maintaining a database of items associated with one or more skill requirements and a visit duration; maintaining a database of experts associated with one or more skill proficiencies, a location, and a schedule; receiving a request from a consumer for delivery by an expert of one or more items in the database to a consumer address; identifying experts in the database having skill proficiencies matching the skill requirements of the one or more items and available in a timeslot for the visit duration of the one or more items; presenting timeslots for which one or more experts are identified to the consumer and allowing the consumer to select a timeslot; and selecting an expert from among the identified experts in the selected timeslot based on shortest travel time; provided that utilization of the selected expert exceeds a predetermined utilization threshold.

### CROSS-REFERENCE

This application claims the benefit of U.S. Provisional Application No. 63/123,265, filed Dec. 9, 2020, which is hereby incorporated by reference in its entirety herein.

### BACKGROUND

Optimal management of patients with amyotrophic lateral sclerosis (ALS) and other neuromuscular diseases requires the ability to accurately assess respiratory function. Timely assessment of pulmonary function is important in gauging prognosis and instituting care such as noninvasive ventilatory support. Approximately half of ALS patients in the United States receive care in multidisciplinary clinics where vital capacity and other pulmonary function studies are routinely performed. Telemedicine is quickly emerging as an appealing alternative to in-clinic visits as it reduces patient burden and allows for more frequent evaluations. While existing telemedicine solutions allow for efficient real-time communication between patients and doctors, remote objective assessment of patients' pulmonary function requires the use of specialized hardware, namely spirometers. This prevents widespread use and has resulted in a recognized need that innovative solutions are required for low-cost, low-burden, remote assessment of pulmonary function, particularly for vulnerable populations.

### SUMMARY

Disclosed herein are systems, methods, and software for prediction or estimating respiratory functional metrics such as forced vital capacity (FVC) based on speech acoustics or speech data. In some embodiments, the speech acoustics are collected remotely via a mobile app without the need for any additional equipment (e.g. a spirometer). In some embodiments, the systems, methods, and software herein employ a machine learning algorithm trained on acoustic samples from both healthy participants and participants with a disease affecting respiratory or pulmonary function such as, for example, amyotrophic lateral sclerosis (ALS).

Disclosed herein, in one aspect, is a computer-implemented method for evaluating pulmonary function comprising: (a) receiving audio data of a subject; (b) extracting one or more acoustic features from the audio data; (c) analyzing the one or more acoustic features using a predictive algorithm to generate an evaluation of pulmonary function for the subject. In some embodiments, the evaluation of pulmonary function comprises a predicted forced vital capacity, forced expiratory volume, peak expiratory flow, mid-expiratory flow rate, forced inspiratory vital capacity, forced expiratory time, respiration rate, respiration rhythm, respiration quality, pause rate, cough events, maximum phonation time, vocal quality, hypernasality, or any combination thereof. In some embodiments, the audio data is collected remotely using a mobile application. In some embodiments, the audio data is collected without requiring a spirometer. In some embodiments, further comprising providing instructions to the subject to perform a vocal task and collecting the audio data during performance of the vocal task by the subject. In some embodiments, further comprising processing the audio data. In some embodiments, the predictive algorithm comprises a trained machine learning model. In some embodiments, the one or more acoustic features comprises at least one of MPT, measures of pitch, loudness, or vocal quality. In some embodiments, further comprising analyzing one or more demographics features. In some embodiments, the one or more demographics features comprises at least one of age, height, gender, or weight. In some embodiments, the subject is undergoing a clinical trial that comprises the evaluation for pulmonary function. In some embodiments, the subject participates in the clinical trial by providing the audio data remotely through an electronic device comprising a microphone. In some embodiments, further comprising monitoring the pulmonary function of the subject over time based on generating the evaluation of pulmonary function over time. In some embodiments, further comprising providing a software application accessible through a user electronic device to the subject for monitoring the pulmonary function of the subject over time. In some embodiments, the software application is configured to prompt the subject with one or more elicitation tasks and record one or more audio recordings of the subject to obtain the audio data. In some embodiments, the one or more acoustic features comprises one or more features recited in Table 2. In some embodiments, the evaluation of pulmonary function comprises a status of a disease area associated with pulmonary function. In some embodiments, the disease area is a respiratory condition or disease affecting bloody oxygenation or homeostasis or a neurodegenerative disease affecting pulmonary function. In some embodiments, the disease area comprises ALS, COPD, asthma, cystic fibrosis, COVID-19, In some embodiments, the audio data is received from an electronic device used by the subject to collect the audio data remotely. In some embodiments, the electronic device is not a specialized device for measuring respiratory function. In some embodiments, the electronic device is a portable user electronic device. In some embodiments, the electronic device is a mobile phone, a smartphone, a tablet, a desktop computer, or a laptop computer. In some embodiments, further comprising providing a software application accessible or downloadable by the electronic device to enable remote collection of the audio data. In some embodiments, the software application is a mobile application. In some embodiments, the mobile application is configured to provide clinical trial registration. In some embodiments, the subject is undergoing one or more digital therapeutics for improving or treating pulmonary function or mitigating or slowing progression of decline of pulmonary function of the subject. In some embodiments, further comprising providing one or more digital therapeutics for improving or treating pulmonary function or mitigating or slowing progression of decline of pulmonary function of the subject. In some embodiments, the one or more digital therapeutics comprises one or more physical therapy exercises for improving pulmonary function. In some embodiments, the one or more digital therapeutics are provided through an integrated software application that provides the one or more digital therapeutics in combination with the evaluation of pulmonary function. In some embodiments, further comprising providing a rewards system for incentivizing subject compliance with the evaluation of pulmonary function and/or the one or more digital therapeutics. In some embodiments, the predictive algorithm generates evaluations of pulmonary function with a correlation coefficient of at least 0.65, 0.70, 0.75, or 0.80 when comparing predicted evaluations to ground truth evaluations for at least 100 independent samples.

Disclosed herein, in another aspect, is a system for evaluating pulmonary function comprising a processor and non-transitory computer readable storage medium comprising computer executable instructions that, when executed by the processor, cause the processor to: (a) receive audio data of a subject; (b) extract one or more acoustic features from the audio data; (c) analyze the one or more acoustic features using a predictive algorithm to generate an evaluation of pulmonary function for the subject. In some embodiments, the evaluation of pulmonary function comprises a predicted forced vital capacity, forced expiratory volume, peak expiratory flow, mid-expiratory flow rate, forced inspiratory vital capacity, forced expiratory time, respiration rate, respiration rhythm, respiration quality, pause rate, cough events, maximum phonation time, vocal quality, hypernasality, or any combination thereof. In some embodiments, the audio data is collected remotely using a mobile application. In some embodiments, the audio data is collected without requiring a spirometer. In some embodiments, further comprising providing instructions to the subject to perform a vocal task and collecting the audio data during performance of the vocal task by the subject. In some embodiments, further comprising processing the audio data. In some embodiments, the predictive algorithm comprises a trained machine learning model. In some embodiments, the one or more acoustic features comprises at least one of MPT, measures of pitch, loudness, or vocal quality. In some embodiments, further comprising analyzing one or more demographics features. In some embodiments, the one or more demographics features comprises at least one of age, height, gender, or weight. In some embodiments, the subject is undergoing a clinical trial that comprises the evaluation for pulmonary function. In some embodiments, the subject participates in the clinical trial by providing the audio data remotely through an electronic device comprising a microphone. In some embodiments, further comprising monitoring the pulmonary function of the subject over time based on generating the evaluation of pulmonary function over time. In some embodiments, further comprising providing a software application accessible through a user electronic device to the subject for monitoring the pulmonary function of the subject over time. In some embodiments, the software application is configured to prompt the subject with one or more elicitation tasks and record one or more audio recordings of the subject to obtain the audio data. In some embodiments, the one or more acoustic features comprises one or more features recited in Table 2. In some embodiments, the evaluation of pulmonary function comprises a status of a disease area associated with pulmonary function. In some embodiments, the disease area is a respiratory condition or disease affecting bloody oxygenation or homeostasis or a neurodegenerative disease affecting pulmonary function. In some embodiments, the disease area comprises ALS, COPD, asthma, cystic fibrosis, COVID-19, In some embodiments, the audio data is received from an electronic device used by the subject to collect the audio data remotely. In some embodiments, the electronic device is not a specialized device for measuring respiratory function. In some embodiments, the electronic device is a portable user electronic device. In some embodiments, the electronic device is a mobile phone, a smartphone, a tablet, a desktop computer, or a laptop computer. In some embodiments, further comprising providing a software application accessible or downloadable by the electronic device to enable remote collection of the audio data. In some embodiments, the software application is a mobile application. In some embodiments, the mobile application is configured to provide clinical trial registration. In some embodiments, the subject is undergoing one or more digital therapeutics for improving or treating pulmonary function or mitigating or slowing progression of decline of pulmonary function of the subject. In some embodiments, further comprising providing one or more digital therapeutics for improving or treating pulmonary function or mitigating or slowing progression of decline of pulmonary function of the subject. In some embodiments, the one or more digital therapeutics comprises one or more physical therapy exercises for improving pulmonary function. In some embodiments, the one or more digital therapeutics are provided through an integrated software application that provides the one or more digital therapeutics in combination with the evaluation of pulmonary function. In some embodiments, further comprising providing a rewards system for incentivizing subject compliance with the evaluation of pulmonary function and/or the one or more digital therapeutics. In some embodiments, the predictive algorithm generates evaluations of pulmonary function with a correlation coefficient of at least 0.65, 0.70, 0.75, or 0.80 when comparing predicted evaluations to ground truth evaluations for at least 100 independent samples.

Disclosed herein, in another aspect, is non-transitory computer readable storage medium comprising computer executable instructions, when executed by a processor, cause the processor to: (a) receive audio data of a subject; (b) extract one or more acoustic features from the audio data; (c) analyze the one or more acoustic features using a predictive algorithm to generate an evaluation of pulmonary function for the subject. In some embodiments, the evaluation of pulmonary function comprises a predicted forced vital capacity, forced expiratory volume, peak expiratory flow, mid-expiratory flow rate, forced inspiratory vital capacity, forced expiratory time, respiration rate, respiration rhythm, respiration quality, pause rate, cough events, maximum phonation time, vocal quality, hypernasality, or any combination thereof. In some embodiments, the audio data is collected remotely using a mobile application. In some embodiments, the audio data is collected without requiring a spirometer. In some embodiments, further comprising providing instructions to the subject to perform a vocal task and collecting the audio data during performance of the vocal task by the subject. In some embodiments, further comprising processing the audio data. In some embodiments, the predictive algorithm comprises a trained machine learning model. In some embodiments, the one or more acoustic features comprises at least one of MPT, measures of pitch, loudness, or vocal quality. In some embodiments, further comprising analyzing one or more demographics features. In some embodiments, the one or more demographics features comprises at least one of age, height, gender, or weight. In some embodiments, the subject is undergoing a clinical trial that comprises the evaluation for pulmonary function. In some embodiments, the subject participates in the clinical trial by providing the audio data remotely through an electronic device comprising a microphone. In some embodiments, further comprising monitoring the pulmonary function of the subject over time based on generating the evaluation of pulmonary function over time. In some embodiments, further comprising providing a software application accessible through a user electronic device to the subject for monitoring the pulmonary function of the subject over time. In some embodiments, the software application is configured to prompt the subject with one or more elicitation tasks and record one or more audio recordings of the subject to obtain the audio data. In some embodiments, the one or more acoustic features comprises one or more features recited in Table 2. In some embodiments, the evaluation of pulmonary function comprises a status of a disease area associated with pulmonary function. In some embodiments, the disease area is a respiratory condition or disease affecting bloody oxygenation or homeostasis or a neurodegenerative disease affecting pulmonary function. In some embodiments, the disease area comprises ALS, COPD, asthma, cystic fibrosis, COVID-19, In some embodiments, the audio data is received from an electronic device used by the subject to collect the audio data remotely. In some embodiments, the electronic device is not a specialized device for measuring respiratory function. In some embodiments, the electronic device is a portable user electronic device. In some embodiments, the electronic device is a mobile phone, a smartphone, a tablet, a desktop computer, or a laptop computer. In some embodiments, further comprising providing a software application accessible or downloadable by the electronic device to enable remote collection of the audio data. In some embodiments, the software application is a mobile application. In some embodiments, the mobile application is configured to provide clinical trial registration. In some embodiments, the subject is undergoing one or more digital therapeutics for improving or treating pulmonary function or mitigating or slowing progression of decline of pulmonary function of the subject. In some embodiments, further comprising providing one or more digital therapeutics for improving or treating pulmonary function or mitigating or slowing progression of decline of pulmonary function of the subject. In some embodiments, the one or more digital therapeutics comprises one or more physical therapy exercises for improving pulmonary function. In some embodiments, the one or more digital therapeutics are provided through an integrated software application that provides the one or more digital therapeutics in combination with the evaluation of pulmonary function. In some embodiments, further comprising providing a rewards system for incentivizing subject compliance with the evaluation of pulmonary function and/or the one or more digital therapeutics. In some embodiments, the predictive algorithm generates evaluations of pulmonary function with a correlation coefficient of at least 0.65, 0.70, 0.75, or 0.80 when comparing predicted evaluations to ground truth evaluations for at least 100 independent samples.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic diagram depicting a system for assessing parameters of speech for upstream and downstream analyses, per one or more embodiments herein;

FIG. 2 is a flow diagram illustrating a series of audio pre-processing steps, feature extraction, and analysis, per one or more embodiments herein;

FIG. 3A and FIG. 3B display individual trajectories of participants from the training set for FVC (blue) and MPT, per one or more embodiments herein;

FIG. 4A shows predicted and observed FVC in a scatterplot (using only the original single overlapping MPT and FVC without any averaging), per one or more embodiments herein;

FIG. 4B and FIG. 4C show the predicted and observed FVC scatterplots when using the average of 3 and 5 MPT measurements, per one or more embodiments herein;

FIG. 5 shows the observed data for the FVC and predicted FVC side-by-side, per one or more embodiments herein;

FIG. 6 shows a non-limiting example of a computing device; in this case, a device with one or more processors, memory, storage, and a network interface, per one or more embodiments herein;

FIG. 7 shows a non-limiting example of a web/mobile application provision system; in this case, a system providing browser-based and/or native mobile user interfaces, per one or more embodiments herein; and

FIG. 8 shows a non-limiting example of a cloud-based web/mobile application provision system; in this case, a system comprising an elastically load balanced, auto-scaling web server and application server resources as well synchronously replicated databases, per one or more embodiments herein.

### DETAILED DESCRIPTION

Patient respiratory health can be evaluated through various respiratory measures including forced vital capacity, forced expiratory volume, respiration rate, and other measures listed in Table 1. The traditional process for obtaining these respiratory measures employs a spirometer or other specialized device such as a peak flow meter. For example, a peak flow meter is often used to measure peak expiratory flow, and windmill-type spirometers can be used for measuring forced vital capacity. Many clinical trials require that the subjects employ a spirometer to collect pulmonary data, such as vital capacity. Additionally, patients may track their respiratory abilities during recovery or treatment and/or to maintain a healthy lifestyle. Such spirometers, however, are impedingly expensive, large, messy to use, and often require proper training or supervision by a healthcare clinician for accurate results. Further, current clinical trials and clinical treatment regimens require patients to obtain a spirometer or travel to a point of care for testing, and to manually enter or transmit the results of such tests. Such drawbacks often reduce the frequency at which such respiratory data can be collected and increase patient/subject non-compliance.

As such, disclosed herein are systems, methods, and software for prediction or estimating forced vital capacity (FVC) based on speech acoustics or speech data, which address a number of deficiencies in the conventional approaches. The systems, methods, and software herein employ a patient's/subject's own smartphone, computer, and/or tablet, thus reducing costs, the need for equipment maintenance, and often ineffective training. Further, the systems, methods, and software herein eliminate the requisite for a patient/subject to travel to a point of care and to manually enter or transmit the results of such tests. As such, the systems, methods, and software herein enable increased testing frequency and patient/subject compliance.

Provided herein are tools for predicting and/or estimating respiratory measures such as FVC which do not require specialized hardware or additional equipment, such as a spirometer. The systems, methods, and software herein employ speech-based tasks (e.g., elicitation tasks such as those listed in Table 3) such as single breath count and maximum sustained phonation collected via a mobile and/or computer application to determine one or more respiratory measures (e.g., FVC), and aid in the diagnosis and/or treatment of asthma and chronic obstructive pulmonary disease (COPD).

In some embodiments, the prediction and/or estimation is determined algorithmically based on speech acoustics and/or audio data obtained from an individual. The speech acoustics and/or audio data can be measured passively (e.g., via passive listening by a microphone of a user device such as a smartphone) or actively (e.g., via the microphone of a smartphone after prompting the user with an elicitation task). In some embodiments, the algorithm is a machine learning algorithm that is trained on data from healthy participants and participants with amyotrophic lateral sclerosis (ALS). In some embodiments, the systems, methods, and software herein predict/estimate a subject's respiratory measure(s) such as FVC and/or associated respiratory health status (e.g., status of disease as reflected in respiratory measure(s)).

An example of a respiratory measure is forced vital capacity, which is a key measurement to assess respiratory function. Forced vital capacity is measured as the volume of air a person can exhale after a forced breath. Forced vital capacity is typically measured by a wet or regular spirometer. A normal adult has a forced vital capacity between 3 and 5 liters. A human's vital capacity can depend on various factors including age, sex, height, and mass. The systems, methods, and software herein exhibit an exceptionally high cross-sectional accuracy and sensitivity to within-subject change, wherein predicted and observed FVC values in the test sample had a correlation coefficient of 0.80 and mean absolute error between 0.54 L and 0.58 L (18.5% to 19.5%). The systems, methods, and software herein are further able to detect longitudinal decline in FVC with a repeatability of 0.92-0.94).

In some embodiments, the systems, methods, and software disclosed herein provide evaluation of pulmonary function via one or more respiratory measures at a high degree of predictive accuracy. When compared to ground truth measurements, the predicted respiratory measures generated based on speech acoustics or audio data are calculated at or above a statistical threshold. For example, the correlation coefficient between a predicted and observed respiratory measure value may be determined by running the algorithm against a set of independent samples. In some instances, the value of a respiratory measure has a correlation coefficient of at least 0.60, 0.65, 0.70, 0.75, 0.80, 0.85, 0.90, or at least 0.95.

### Machine Learning Training Data

In some embodiments, machine learning training data is used to train a suitable model for evaluating one or more respiratory measures based on speech acoustics and/or audio data (e.g., audio samples). In one non-limiting example, machine learning training data was collected from two separate sources, a first source wherein home-collected audio data was used to train a machine learning model to determine FVC (training data), and a second source wherein home-collected audio data was used to test the machine learning model's accuracy in determining FVC (validation of the trained model). In some embodiments, the first source and the second source comprise audio samples and corresponding spirometry measurements (e.g. by an AirSmart Spirometer—Nuvoair AB, Stockholm, Sweden). In some embodiments, the first study and the second study further comprise a physical health parameter. In some embodiments, the physical health parameter comprises a grip strength, a general activity level, an electrical impedance myography, a ALS Functional Rating Scale test result, a spirometry reading, or any combination thereof.

In some embodiments, the audio samples comprise a subject's response to a maximum-effort sustained phonation task. In some embodiments, the audio samples further comprises a period of collected ambient. To perform the maximum-effort sustained phonation task, subjects were instructed to take a deep breath and say “ahh” for as long as possible until they ran out of breath. Various elicitation tasks that can be used to obtain the audio samples are contemplated, including without limitation, any combination of the examples recited in Table 3.

The collection of audio samples for training the machine learning model can take place over a duration of time to improve accuracy of the model by accounting for variation over time. In one example, the first source and the second source comprise three months of daily audio files and six months of twice-weekly audio files, per the table below.

| Description | First Source | | Second Source | |
|---|---|---|---|---|
| Number of Participants | 33 ALS; 6 HC | | 25 ALS | |
| Total Observations | 1,971 (overlapping | | 47 FVC obs. | |
| | FVC and sus. phon.) | | 578 sus. phon. | |
| Gender | 15 F; 24 M | | 8 F; 17 M | |
| Age (mean, SD) | 58.1 | (SD = 9.4) | 65.4 | (SD = 11.8) |
| FVC (L) (mean, SD) | 3.51 | (SD = 1.15) | 3.38 | (SD = 1.31) |
| ALSFRS-R total (in ALS | 37.1 | (SD = 7.3) | 36.9 | (SD = 6.0) |
| participants) | | | | |
| Height (cm) | 172.0 | (8.6) | 173.8 | (SD = 11.5) |

### Sample Descriptive Statistics

### Systems for Collecting Audio Samples

In some embodiments, the audio samples from the first source, the second source, or both were collected by a system 100 for collecting audio samples.

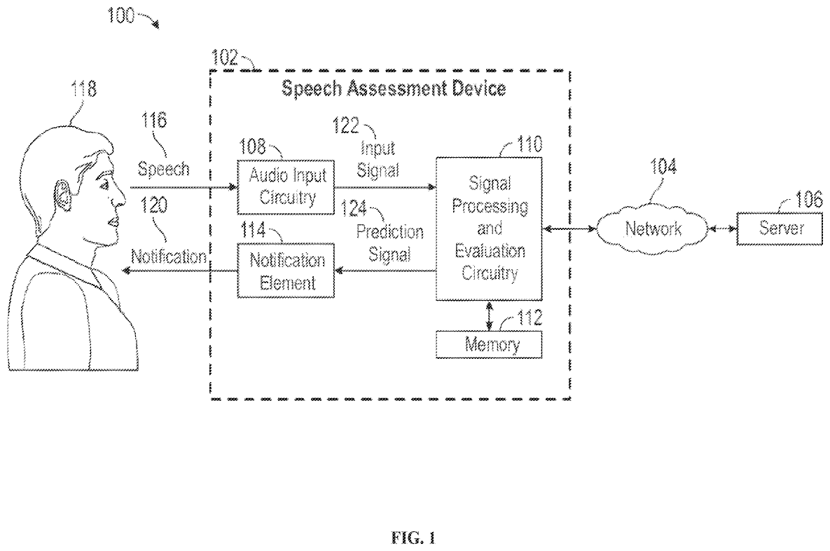

FIG. 1 is a diagram of a system 100 for collecting audio samples, the system comprising an audio sample collection device 102, a network 104, and a server 106. The audio sample collection device 102 comprises audio input circuitry 108, signal processing circuitry 110, memory 112, and at least one notification element 114. In certain embodiments, the signal processing circuitry 110 comprises audio processing circuitry. In some cases, the signal processing circuitry is configured to provide at least one speech assessment signal (e.g. generated outputs based on algorithmic/model analysis of input feature measurements) based on characteristics of speech provided by a user (e.g. speech or audio stream or data). In some embodiments, the audio input circuitry 108, notification element(s) 114, and memory 112 are coupled with the signal processing circuitry 110 via wired connections, wireless connections, or a combination thereof. In some embodiments, the audio sample collection device 102 comprises a smartphone, a smartwatch, a wearable sensor, a computing device, a headset, a headband, or combinations thereof. The audio sample collection device 102 in some embodiments, is configured to receive speech 116 from a user 118 and provide a notification 120 to the user 118 based on processing the speech 116 and any associated signals to generate output corresponding to upstream or downstream analyses using one or more trained models relating to pulmonary diseases and/or measurements. In some cases, such analyses are associated with ALS, COPD, asthma, cystic fibrosis, and other pulmonary diseases. For example, one or more respiratory measures may be used as the status indicator(s) for a pulmonary disease.

In some embodiments, the audio input circuitry 108 comprises at least one microphone. In certain embodiments, the audio input circuitry 108 comprises a bone conduction microphone, a near field air conduction microphone array, or a combination thereof. In some embodiments, the audio input circuitry 108 is configured to provide an input signal 122 that is indicative of the speech 116 provided by the user 118 to the signal processing circuitry 110. In some embodiments, the input signal 122 is formatted as a digital signal, an analog signal, or a combination thereof. In certain embodiments, the audio input circuitry 108 provides the input signal 122 to the signal processing circuitry 110 over a personal area network (PAN). The PAN in some embodiments comprises Universal Serial Bus (USB), IEEE 1394 (FireWire) Infrared Data Association (IrDA), Bluetooth, ultra-wideband (UWB), Wi-Fi Direct, or a combination thereof. The audio input circuitry 108 in some embodiments further comprise at least one analog-to-digital converter (ADC) to provide the input signal 122 in digital format.

In some embodiments, the signal processing circuitry 110 comprises a communication interface (not shown) coupled with the network 104 and a processor (e.g. an electrically operated microprocessor (not shown) configured to execute a pre-defined and/or a user-defined machine readable instruction set, such as may be embodied in computer software) configured to receive the input signal 122. In some embodiments, the communication interface comprises circuitry for coupling to the PAN, a local area network (LAN), a wide area network (WAN), or a combination thereof. In some embodiments, the processor is configured to receive instructions (e.g. software, that is periodically updated) for extracting and/or computing one or more speech or acoustic features relevant to downstream analysis of a pulmonary disease or functionality of the user 118.

In certain embodiments, the processor comprises an ADC to convert the input signal 122 to digital format. In other embodiments, the processor is configured to receive the input signal 122 from the PAN via the communication interface. In some embodiments, the processor further comprises level detect circuitry, adaptive filter circuitry, voice recognition circuitry, or any combination thereof. The processor in some embodiments is further configured to process the input signal 122 using one or more metrics or features derived from a speech input signal and produce a speech/audio assessment signal (e.g., indicative of one or more respiratory measures), and optionally provide a prediction signal (e.g., of respiratory function) 124 to the notification element 114. The prediction signal 124 in some embodiments is in a digital format, an analog format, or a combination thereof. In certain embodiments, the prediction signal 124 comprises one or more of an audible signal, a visual signal, a vibratory signal, or another user-perceptible signal. In certain embodiments, the processor additionally or alternatively provides the prediction signal 124 over the network 104 via a communication interface.

The processor in some embodiments is further configured to generate a record indicative of the prediction signal 124. In some embodiments, the record comprises a sample identifier and/or an audio segment indicative of the speech 116 provided by the user 118. In certain embodiments, the user 118 is prompted to provide current symptoms or other information about their current well-being to the audio sample collection device 102 for assessing language and/or acoustic elements of the audio/speech and associated respiratory measures such as FVC or other pulmonary data. Such information may be included in the record, and may further be used to aid in identification or further prediction of changes in respiratory measures such as FVC or associated pulmonary conditions.

In some embodiments, the record further comprises a location identifier, a time stamp, a physiological sensor signal (e.g. heart rate, blood pressure, temperature, or the like), or a combination thereof being correlated to and/or contemporaneous with the speech signal 124. The location identifier in some embodiments comprises a Global Positioning System (GPS) coordinate, a street address, a contact name, a point of interest, or a combination thereof. In certain embodiments, a contact name is derived from the GPS coordinate and a contact list associated with the user 118. The point of interest in some embodiments is derived from the GPS coordinate and a database including a plurality of points of interest. In certain embodiments, the location identifier is a filtered location for maintaining the privacy of the user 118. For example, the filtered location may be “user's home”, “contact's home”, “vehicle in transit”, “restaurant”, or “user's work”. In certain embodiments, the record includes a location type, wherein the location identifier is formatted according to the location type.

In some embodiments, the processor is further configured to store the record in the memory 112. The memory 112 may be a non-volatile memory, a volatile memory, or a combination thereof. In some embodiments, the memory 112 is wired to the signal processing circuitry 110 using an address/data bus. In certain embodiments, the memory 112 is a portable memory coupled with the processor.

In certain embodiments, the processor is further configured to send the record to the network 104, wherein the network 104 sends the record to the server 106. In certain embodiments, the processor is further configured to append to the record a device identifier, a user identifier, or a combination thereof. The device identifier in some embodiments is unique to the audio sample collection device 102. The user identifier in some embodiments is unique to the user 118. In some embodiments, the device identifier and the user identifier are useful to a medical treatment professional and/or researcher, wherein the user 118 in some embodiments is a patient of the medical treatment professional.

The network 104 may comprise a PAN, a LAN, a WAN, or a combination thereof. The PAN may comprise USB, IEEE 1394 (FireWire) IrDA, Bluetooth, UWB, Wi-Fi Direct, or a combination thereof. The LAN may include Ethernet, 802.11 WLAN, or a combination thereof. The network 104 may also include the Internet.

The server 106 may comprise a personal computer (PC), a local server connected to the LAN, a remote server connected to the WAN, or a combination thereof. In certain embodiments, the server 106 is a software-based virtualized server running on a plurality of servers.

In certain embodiments, at least some signal processing tasks is performed via one or more remote devices (e.g. the server 106) over the network 104 instead of within an audio sample collection device 102 that houses the audio input circuitry 108.

In certain embodiments, an audio sample collection device 102 comprises a mobile application configured to run on a mobile computing device (e.g. smartphone, smartwatch) or another computing device. With a mobile application, speech samples can be collected remotely from patients and analyzed without requiring patients to visit a clinic. A user 118 in some embodiments is periodically queried (e.g. two, three, four, five, or more times per day) to provide a speech sample, for example, by actively requesting performance of a task. For example, the notification element 114 in some embodiments is used to prompt the user 118 to provide speech/audio 116 from which the input signal 122 is derived, such as through a display message or an audio alert. The notification element 114 may further provide instructions to the user 118 for providing the speech 116 (e.g. displaying a passage for the user 118 to read). In certain embodiments, the notification element 114 may request current symptoms or other information about the current well-being of the user 118 to provide additional data for analyzing the speech 116. Alternatively, or in combination, speech/audio can be passively collected through a user's phone as they go about their day.

In certain embodiments, a notification element may include a display (e.g. LCD display) that displays text and prompts the user to read the text. Each time the user provides a new sample using the mobile application, one or more features (e.g. elemental components of the speech/audio such as language components and acoustic components) of the user's speech abilities in some embodiments is automatically extracted and/or computed. For example, certain audio features may require further algorithmic computation to calculate a feature useful for determining FVC.

In certain embodiments, a user may download a mobile application to a personal computing device (e.g. smartphone), optionally sign into the application, and follow the prompts on a display screen. The prompts may provide one or more elicitation tasks to obtain audio recording(s) of the user in response to said tasks. Once recording has finished, the audio data in some embodiments is automatically uploaded to a secure server (e.g. a cloud server or a traditional server) where the signal processing and machine learning algorithms operate on the recordings. Alternatively, one or more of the signal processing and machine learning algorithms are integrated with the local or remote software application on the user device (e.g., smartphone) to enable audio data processing and/or analysis to generate predictions or estimates of respiratory measure(s) without requiring network access.

### Speech and Audio Acquisition and Analysis

Disclosed herein are speech and audio acquisition and analysis algorithms that enable the acquisition, processing and/or feature extraction, and analysis of audio features to generate estimates or predictions of various respiratory measures such as forced vital capacity. In some embodiments, a speech analysis algorithm determines a maximum phonation time (MPT) based on the collected ambient noise sample and sustained phonation. MPT is measured as a length of the sustained phonation from phonation onset to phonation offset. The speech analysis algorithms herein are capable of calculating MPT within a mean absolute error of 0.01 seconds, as determined by a comparison of automatically-calculated MPT values to ground truth MPT values computed from 100 randomly selected speech files, where speech onset and offset were manually labeled by trained annotators.

In one non-limiting example, a mixed-effects machine learning model was constructed to predict the respiratory measure FVC from the relevant features of height, age, and MPT. Cross-sectionally, this model had a maximum out-of-sample accuracy of 0.54 L MAE (18.5% relative MAE) with a correlation between the predicted and observed FVC values of r=0.80. To predict longitudinal change, a growth curve model was fit to observed and predicted FVC. The slope of the predicted FVC was slightly less steep than the slope of the observed FVC for the test sample. There are two possible explanations for this. First, the model was trained using at-home spirometry measures whereas the test sample used in-clinic spirometry measures. Second, participants performed at-home spirometry without the guidance of a clinician, whereas the in-clinic spirometry was administered by a respiratory therapist according to standard protocol.

The repeatability of the FVC prediction was quite good, though slightly lower than the repeatability of the observed FVCs. This was a result of the lower reliability of the MPT measurements, also observed in other studies. Several studies have analyzed how to elicit maximum-performance sustained phonation in other contexts and have suggested that modeling and repeat performance of the sustained phonation task increases MPT and improves reliability. Future studies that aim to assess FVC via MPT would benefit from modeling the sustained phonation via training videos and then repeating performance of the task for each session and taking the maximum among the tasks.

Unlike a standard FVC test, the sustained phonation is modulated by both the vital capacity and the valving of the column of exhaled air by the vocal folds. Thus, maximum phonation time is impacted both by phonatory function and respiratory function. This is an important consideration in the present study, as vocal quality may change over time in ALS, especially in the case of bulbar-onset. To explore the relative contributions of phonatory and respiratory function to MPT, two models were fit using FVC as a proxy for respiration and cepstral peak prominence (CPP) as a proxy for phonation quality, and for each model, the amount of variation explained by the predictor was determined using the R² for mixed-effects models. In the first model, MPT was predicted based on FVC and found that R²=0.24. Then a model was fit where the MPT was predicted based on CPP alone and found that R²=0.01. Therefore, the variability in MPT was moderately influenced by respiration, but only mildly influenced by the vocal quality of the phonatory function. The strong association between FVC and predicted FVC in our results also support MPT as a measure for respiratory function, with only minimal impact from the quality of the phonation for these participants. It would be interesting to determine whether specific clinical characteristics such as bulbar burden contributed to reliability and/or predictive accuracy, but patient numbers were not sufficient to assess this.

MPT is only one of a number of tasks that might be predictive of VC; it will be up to future studies to determine whether MPT can function in a useful manner and if other tasks may provide equivalent or improved predictive capacity. The extent to which MPT might serve as a useful outcome measure in clinical trials is an important question. The natural history cohorts that form the basis of this study were not selected with clinical trial inclusion criteria in mind; future studies in cohorts more representative of the clinical trial population will help to determine how this measure functions in that environment. Further experience in the clinical setting will also help determine the extent to which MPT can serve as a clinically useful surrogate in clinical situations where VC cannot be obtained, either because a visit is being conducted remotely or if the procedure is deemed a risk for any reason.

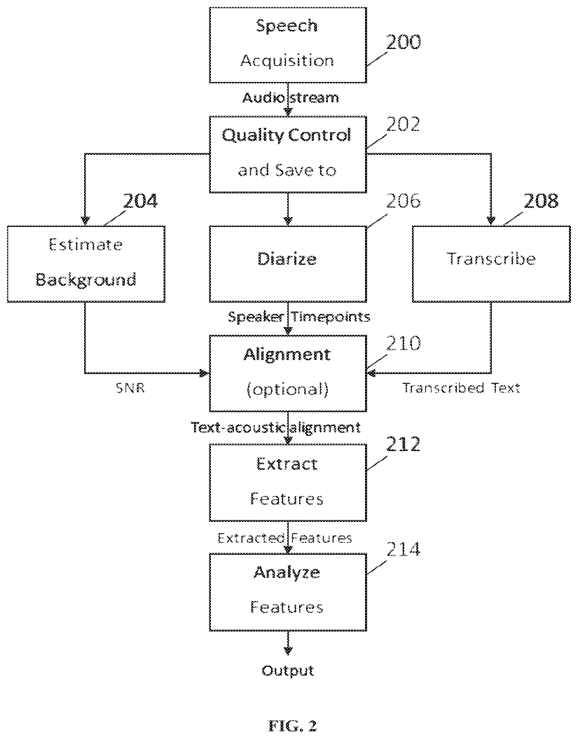

As shown in FIG. 2, the process for speech/language feature extraction and analysis can include one or more steps such as speech acquisition 200, quality control 202, background noise estimation 204, diarization 206, transcription 208, optional alignment 210, feature extraction 212, and/or feature analysis 214. In some embodiments, the systems, devices, and methods disclosed herein include a speech acquisition step. Speech acquisition 200 can be performed using any number of audio collection devices. Examples include microphones or audio input devices on a laptop or desktop computer, a portable computing device such as a tablet, mobile devices (e.g. smartphones), digital voice recorders, audiovisual recording devices (e.g. video camera), and other suitable devices. In some cases, these devices are configured with software to provide digital therapeutics, for example, cognitive behavioral therapy. In some embodiments, the speech or audio is acquired through passive collection techniques. For example, a device in some embodiments passively collects background speech via a microphone without actively eliciting the speech from a user or individual. The device or software application implemented on the device in some embodiments is configured to begin passive collection upon detection of background speech. Alternatively, or in combination, speech acquisition can include active elicitation of speech. For example, a mobile application implemented on the device may include instructions prompting speech by a user or individual. In some embodiments, the user is prompted to provide conversational responses to questions or a verbal description such as, for example, a picture description. In some embodiments, the systems, devices, and methods disclosed herein utilize a dialog bot or chat bot that is configured to engage the user or individual in order to elicit speech. As an illustrative example, the bot may engage in a conversation with the user (e.g. via a graphic user interface such as a smartphone touchscreen or via an audio dialogue). Alternatively or in combination with a conversation, the bot may simply provide instructions to the user to perform a particular task (e.g. instructions to vocalize pre-written speech or sounds). In some cases, the speech or audio is not limited to spoken words, but can include nonverbal audio vocalizations made by the user or individual. For example, the user in some embodiments is prompted with instructions to make a sound that is not a word for a certain duration.

In some embodiments, the systems, devices, and methods disclosed herein include a quality control step 202. The quality control step may include an evaluation or quality control checkpoint of the speech or audio quality. Quality constraints in some embodiments are applied to speech or audio samples to determine whether they pass the quality control checkpoint. Examples of quality constraints include (but are not limited to) signal to noise ratio (SNR), speech content (e.g. whether the content of the speech matches up to a task the user was instructed to perform), audio signal quality suitability for downstream processing tasks (e.g. speech recognition, diarization, etc.). Speech or audio data that fails this quality control assessment in some embodiments is rejected, and the user asked to repeat or redo an instructed task (or alternatively, continue passive collection of audio/speech). Speech or audio data that passes the quality control assessment or checkpoint in some embodiments is saved on the local device (e.g. user smartphone, tablet, or computer) and/or on the cloud. In some cases, the data is both saved locally and backed up on the cloud. In some embodiments, one or more of the audio processing and/or analysis steps are performed locally or remotely on the cloud.

In some embodiments, the systems, devices, and methods disclosed herein include background noise estimation 204. Background noise estimation can include metrics such as a signal-to-noise ratio (SNR). SNR is a comparison of the amount of signal to the amount background noise, for example, ratio of the signal power to the noise power in decibels. Various algorithms can be used to determine SNR or background noise with non-limiting examples including data-aimed maximum-likelihood (ML) signal-to-noise ratio (SNR) estimation algorithm (DAML), decision-directed ML SNR estimation algorithm (DDML) and an iterative ML SNR estimation algorithm.

In some embodiments, the systems, devices, and methods disclosed herein perform audio analysis of speech/audio data stream such as speech diarization 206 and speech transcription 208. The diarization process can include speech segmentation, classification, and clustering. In some cases when there is only one speaker, diarization is optional. The speech or audio analysis can be performed using speech recognition and/or speaker diarization algorithms Speaker diarization is the process of segmenting or partitioning the audio stream based on the speaker's identity. As an example, this process can be especially important when multiple speakers are engaged in a conversation that is passively picked up by a suitable audio detection/recording device. In some embodiments, the diarization algorithm detects changes in the audio (e.g. acoustic spectrum) to determine changes in the speaker, and/or identifies the specific speakers during the conversation. An algorithm in some embodiments is configured to detect the change in speaker, which can rely on various features corresponding to acoustic differences between individuals. The speaker change detection algorithm may partition the speech/audio stream into segments. These partitioned segments may then be analyzed using a model configured to map segments to the appropriate speaker. The model can be a machine learning model such as a deep learning neural network. Once the segments have been mapped (e.g. mapping to an embedding vector), clustering can be performed on the segments so that they are grouped together with the appropriate speaker(s).

Techniques for diarization include using a Gaussian mixture model, which can enable modeling of individual speakers that allows frames of the audio to be assigned (e.g. using Hidden Markov Model). The audio can be clustered using various approaches. In some embodiments, the algorithm partitions or segments the full audio content into successive clusters and progressively attempts to combine the redundant clusters until eventually the combined cluster corresponds to a particular speaker. In some embodiments, algorithm begins with a single cluster of all the audio data and repeatedly attempts to split the cluster until the number of clusters that has been generated is equivalent to the number of individual speakers. Machine learning approaches are applicable to diarization such as neural network modeling. In some embodiments, a recurrent neural network transducer (RNN-T) is used to provide enhanced performance when integrating both acoustic and linguistic cues. Examples of diarization algorithms are publicly available (e.g. Google).

Speech recognition (e.g. transcription of the audio/speech) in some embodiments is performed sequentially or together with the diarization. The speech transcript and diarization can be combined to generate an alignment of the speech to the acoustics (and/or speaker identity). In some cases, passive and active speech are evaluated using different algorithms. Standard algorithms that are publicly available and/or open source in some embodiments is used for passive speech diarization and speech recognition (e.g. Google and Amazon open source algorithms) Non-algorithmic approaches can include manual diarization. In some embodiments, diarization and transcription are not required for certain tasks. For example, the user or individual in some embodiments are instructed or required to perform certain tasks such as sentence reading tasks or sustained phonation tasks in which the user is supposed to read a pre-drafted sentence(s) or to maintain a sound for an extended period of time. In such tasks, transcription may not be required because the user is being instructed on what to say. Alternatively, certain actively acquired audio in some embodiments is analyzed using standard (e.g. non-customized) algorithms or, in some cases, customized algorithms to perform diarization and/or transcription. In some embodiments, the dialogue or chat bot is configured with algorithm(s) to automatically perform diarization and/or speech transcription while interacting with the user

In some embodiments, the speech or audio analysis comprises alignment 210 of the diarization and transcription outputs. The performance of this alignment step may depend on the downstream features that need to be extracted. For example, certain features require the alignment to allow for successful extraction (e.g. features based on speaker identity and what the speaker said), while others do not. In some embodiments, the alignment step comprises using the diarization output to extract the speech from the speaker of interest. Standard algorithms in some embodiments are used with non-limiting examples including Kaldi, gentle, Montreal forced aligner), or customized alignment algorithms (e.g. using algorithms trained with proprietary data).

In some embodiments, the systems, devices, and methods disclosed herein perform feature extraction 212 from one or more of the SNR, diarization, and transcription outputs. One or more extracted features can be analyzed 214 to predict or determine an output comprising one or more composites or related indicators of speech production. In some embodiments, the output comprises an indicator of a physiological condition such as a respiratory status or impairment (e.g. current status of ALS, COPD, asthma, or cystic fibrosis as measured by one or more respiratory measures).

The systems, devices, and methods disclosed herein may implement or utilize a plurality or chain or sequence of models or algorithms for performing analysis of the features extracted from a speech or audio signal. In some embodiments, the plurality of models comprises multiple models individually configured to generate specific composites or perceptual dimensions. In some embodiments, one or more outputs of one or more models serve as input for one or more next models in a sequence or chain of models. In some embodiments, one or more features and/or one or more composites are evaluated together to generate an output. In some embodiments, a machine learning algorithm or ML-trained model (or other algorithm) is used to analyze a plurality of feature or feature measurements/metrics extracted from the speech or audio signal to generate an output such as a composite. In some embodiments, the systems, devices, and methods disclosed herein combine the features to produce one or more composites that describe or correspond to an outcome, estimation, or prediction, for example, corresponding to pulmonary diseases or capabilities.

In some embodiments, the respiratory measures in Table 1 and Table 2 below are estimated based on a video or audio of a patient/subject performing one or more of the elicitation tasks in Table 3. In some embodiments, the patient/subject is elicited to perform the one or more elicitation tasks using a stand-alone mobile application, by an embedded analysis platform within a 3rd party product (e.g. a digital therapeutic), or both.

In some embodiments, a first set of video/audio files with associated respiratory measurements, is used to train a machine learning algorithm to determine an estimated respiratory measurement of a second sent of video/audio files. In some embodiments, a unique model is built for each respiratory measurement. In some embodiments, a unique model is built for each respiratory measurements. In some embodiments, additional patient/subject measurements and variables are elicited and used to train the machine learning algorithms. In some embodiments, the additional patient/subject measurements and variables comprise a spirometer measurement.

To build a model, data can be collected from many subjects from a clinical population of interest and healthy controls. In one example, data collection included both acoustic data (elicited using one or more of the elicitations listed above) and ground truth respiratory measures using a spirometer and/or other accepted clinical measures. In some embodiments, the machine learning model is a simple linear model (e.g. variants of linear/logistic regression). In some embodiments, the machine learning model is a complex model, such as a support vector regression model or a deep neural network. A specific model for predicting a particular respiratory measure can be generated by training on the acoustic data labeled with the ground truth respiratory measure as measured by the specialized hardware device (e.g., peak flow meter for measuring peak expiratory flow). Table 1 below lists non-limiting examples of respiratory measures that can be determined according to the systems, methods, and media disclosed herein without use of specialized equipment such as a spirometer.

TABLE 1

| Respiratory measure | Definition | Disease areas |
|---|---|---|
| FVC | The maximum volume of air exhaled | ALS, COPD, asthma |
| (Forced Vital Capacity) | forcefully after a maximal inspiration | |
| FEV1 | The volume of air exhaled during the | ALS, COPD, asthma, |
| (Forced Expiratory Volume) | first second of a forced expiratory | cystic fibrosis |
| | maneuver | |
| PEF | The highest instantaneous airflow rate | COPD, asthma, cystic |
| (Peak Expiratory Flow) | measured during the FVC maneuver | fibrosis |
| FEF2575 | The average forced expiratory flow | COPD, asthma, cystic |
| (Mid-expiratory Flow Rate) | during the mid (25-75%) portion of the | fibrosis |
| | FVC maneuver | |
| FIVC | The volume of air taken in during a | COPD, asthma, cystic |
| (Forced Inspiratory Vital | forced inhalation | fibrosis |
| Capacity) | | |
| FET | The length of the expiration in seconds, | COPD, asthma, cystic |
| (Forced Expiratory Time) | measured by ascultation of the trachea | fibrosis |
| | and a stopwatch | |
| Respiration rate | The number of breaths exchanged in | Respiratory and non- |
| | one minute of breathing at rest. | respiratory conditions |
| | | that blood oxygenation |
| | | and homeostasis |
| Respiration rhythm | The tempo of breathing as measured by | Respiratory and non- |
| | the regularity of inter-breath intervals. | respiratory conditions |
| | | that blood oxygenation |
| | | and homeostasis |
| Respiration quality | A clinical assessment of how relaxed | Respiratory and non- |
| | and silent a patient's breathing is. | respiratory conditions |
| | | that blood oxygenation |
| | | and homeostasis |
| Pause rate | A measure of breathlessness; the | Respiratory and non- |
| | amount of time that a speaker pauses | respiratory conditions |
| | for a breath while speaking | that blood oxygenation |
| | | and homeostasis |
| Cough events | A count of the number of coughs that | COPD, asthma, ALS, |
| | occur during a recording | neurodegenerative |
| | | disease |
| Maximum phonation time | The maximum amount of time that a | COPD, asthma, COVID- |
| | sustained vowel sound can be held | 19, ALS, |
| | following maximal inhalation | neurodegenerative |
| | | disease |
| Vocal quality | The vibratory characteristics of the | Any population for which |
| | vocal folds defined perceptually and/or | maximum phonation time |
| | acoustically | is assessed |
| Hypernasality | The abnormally high amount of nasal | Any population for which |
| | resonance indicative of velopharyngeal | maximum phonation time |
| | port dysfunction as measured | is assessed |
| | perceptually and/or acoustically | |

The clinical purpose of each respiratory measurement and its connection to speech and/or acoustic features are additionally described below in Table 2.

TABLE 2

| | | Connection between speech and |
|---|---|---|
| | | acoustic features and the |
| Respiratory measure | Purpose | respiratory measure |
| FVC | Used to monitor respiratory | Speech acoustic features that can |
| (Forced Vital Capacity) | function in ALS; also used, in | contribute to estimation of FVC |
| | combination with FEV1, to | include (but are not limited to) |
| | measure airway obstruction and | maximum speech/phonation time, |
| | lung restriction | speech/phonation energy and its rate |
| | | of change, frequency of pause |
| | | intervals in speech, time-frequency |
| | | representations. |
| FEV1 | In combination with FVC, used in | The same features used for FVC can |
| (Forced Expiratory | diagnosis of airway obstruction | be used here, however they are only |
| Volume) | and lung restriction | calculated for the first second of the |
| | | forced expiratory maneuver. |
| PEF | Used primarily in monitoring | Statistics of acoustic features |
| (Peak Expiratory Flow) | airway obstruction in COPD and | extracted for FVC can be used |
| | asthma | estimation of highest airflow rate. For |
| | | example, the 95th percentile of the |
| | | features used for FVC, estimated |
| | | over the duration of the task. |
| FEF2575 | Used as an index of pulmonary | The same features used for FVC can |
| (Mid-expiratory Flow | ventilatory function in the context | be used here, however they are |
| Rate) | of obstructive pulmonary | calculated during the mid (25%- |
| | disease. Predictive of exercise | 75%) portion of the forced expiratory |
| | intolerance. | maneuver. |
| FIVC | One of multiple measures | Acoustic features that can contribute |
| (Forced Inspiratory Vital | valuable in assessing | to estimation of air volume during |
| Capacity) | effectiveness of bronchodilation | forced inhalation include acoustic |
| | interventions | energy and its rate of change and |
| | | time-frequency representations of |
| | | acoustic signal. |
| FET | Prolonged FET is indicative of | Speech acoustic features that can |
| (Forced Expiratory | obstructive pulmonary disease, | contribute to estimation of FVC |
| Time) | and can be used to differentiate | include (but are not limited to) |
| | obstructive from healthy or | maximum speech/phonation time, |
| | restrictive pulmonary disease. | speech/phonation energy and its rate |
| | | of change. Because duration of the |
| | | expiration in the FET measurement is |
| | | based on the acoustic breath sounds |
| | | through the trachea, this measure is |
| | | conceivably attainable with a |
| | | microphone. |
| Respiration rate | The rate of breathing at rest | Acoustic features that can contribute |
| | (exchanging tidal volume) is | to estimation of respiration rate |
| | unconsciously mediated by | include (but are not limited to) |
| | chemoreceptors that monitor | envelope modulation spectra and |
| | oxygen and carbon dioxide | time-frequency representations. |
| | levels in the blood and | |
| | cerebrospinal fluid. Abnormally | |
| | high or low rates of breathing at | |
| | rest are indicators of | |
| | deterioration. | |
| Respiration rhythm | During rest breathing | Acoustic features that can contribute |
| | (exchanging tidal volume) the | to estimation of respiration rhythm |
| | inhalatory phase is about twice | include (but are not limited to) |
| | as long as the subsequent | envelope modulation spectra and |
| | exhalation, followed by a brief | time-frequency representations. |
| | period of apnea prior to the next | |
| | inhalation. Abnormalities in | |
| | respiratory rhythm are | |
| | suggestive of challenges in | |
| | maintaining homeostasis. | |
| Respiration quality | The clinical impression of | Acoustic features that can contribute |
| | struggling to breathe or hearing | to estimation of respiration quality |
| | breath sounds or phonation with | include (but are not limited to) |
| | breathing are indicative of | envelope modulation spectra and |
| | challenges in maintaining | time-frequency representations. |
| | homeostasis. | |
| Pause rate | Speaking occurs on exhalation | Measures of silent (non-speech) |
| | and inhalation requires cessation | segments associated with inhalations. |
| | of speech. Increased pause rate | |
| | is indicative of the need for | |
| | oxygen. | |
| Cough events | Used as an index of airway | Acoustic features associated with |
| | irritation during speech, and as | ballistic expulsion of air through |
| | an index of possible laryngeal | closed glottis. |
| | penetration following swallowing. | |
| Maximum phonation | Used as a proxy for FVC and | Acoustic features that can contribute |
| time | FEV1 | to estimation of respiration quality |
| | | include (but are not limited to) |
| | | envelope modulation spectra and |
| | | time-frequency representations. |
| Vocal quality | Sustained phonation requires | Acoustic features that capture |
| | valving of the airstream through | symmetry, degree of medial |
| | the trachea by the vibrating | compression, and periodicity of vocal |
| | vocal folds. Abnormalities in | fold vibration, including (but not |
| | valving (too much or too little) | limited to) harmonics to noise ratio, |
| | impact the rate and volume of | cepstral peak prominence, jitter and |
| | exhaled air, irrespective of | shimmer. |
| | expiratory volume. | |
| Hypernasality | Sustained vowel phonation | Acoustic features that capture nasal |
| | requires closure of the | resonance. |
| | velopharyngeal port to direct the | |
| | airflow and acoustic energy | |
| | through the oral cavity. | |
| | Hypernasality is indicative of | |
| | structural or neuromuscular | |
| | impairment of port closure. | |
| | Inadequate closure results in air | |
| | wastage, thereby decreasing | |
| | maximum phonation time | |
| | irrespective of expiratory | |
| | volume. | |

Exemplary elicitation tasks are listed in Table 3 below.

TABLE 3

| Elicitation Tasks | Type of task | Description of task |
|---|---|---|
| Sustained phonation | Forced | The speaker takes a deep breath and then sustains the |
| (vowel only) | expiratory | sound ‘aahhhh’ for as long as possible on the exhalation, |
| | | trying to keep the sound steady. |
| Sustained phonation | Forced | The speaker takes a deep breath and then sustains the |
| (voiced/unvoiced e.g. | expiratory | sound ‘s’ for as long as possible on the exhalation, at a |
| s/z) | | comfortable pitch and loudness. Then the speaker |
| | | repeats this procedure for the sound ‘z’. |
| Counting until you run | Forced | The speaker takes a deep breath and then counts to the |
| out of breath | expiratory | beat of a metronome, at a comfortable pitch and |
| | | loudness, without taking a breath. |
| Normal breathing | Breathing | Prior to the start of the session, at least one minute of |
| | | video of the participant at rest (not talking, not attending |
| | | to breathing) will be collected. Breath rate will be |
| | | calculated as number of visible inhalations (chest rise) |
| | | per minute. |
| Spontaneous or read | Passive/Natural | Immediate story recall and reading passages will be |
| speech | | evaluated for speaking rate and pause rate. |

### FVC Prediction Model

A machine learning (ML) model was trained to predict FVC based on the at-home data. Several acoustic features and demographic characteristics were considered, including MPT, measures of pitch, loudness, and vocal quality extracted from the sustained phonation, and age, height, gender, and weight. A mixed-effects framework was used to account for the repeated measurements per participant. To separate the between-person effects and within-person effects, each feature extracted from the phonation was dis aggregated such that each participant would have a mean for each predictor and a deviation from the mean for each observation, following the within-person effects disaggregation method.

Both linear and nonlinear models consisting of different sets of variables were tested. The performance of each model was evaluated using leave-one-participant-out cross-validation on the training data. The model was estimated on a training sample consisting of all participants minus one, and the outcome was predicted on the participant that was left out of the training sample using the estimated model. This process was repeated leaving out one participant at a time. The performance of each model was evaluated using the mean absolute error (MAE, described below) between the predicted and observed FVC values using the out-of-sample predictions (the estimates obtained in each participant that was left out while training set for the model). The final model was a linear model which included age, height, and MPT as features. The same approach to model building described herein is contemplated for additional respiratory measures such as those listed in Table 1.

In some embodiments, a tool for predicting a respiratory measure such as forced vital capacity is provided without requiring any specialized hardware or additional equipment such as a spirometer. The prediction or estimation can be generated using an algorithm configured to analyze speech acoustics or audio data. The algorithm can comprise a machine learning model trained on a training data set of healthy participants and participants with ALS. Accordingly, the systems, methods, and software utilizing this tool can be used to screen subjects for respiratory health status such as, for example, forced vital capacity values.

### Machine Learning Accuracy and Repeatability

As described herein, it was possible to assess respiratory function using various features such as a maximum-performance phonation task or other features described herein, including without limitation the speech/audio features associated with various respiratory functions as listed in Table 2. This could be done remotely and by using a phone, without the need for specialized equipment. Predicted FVC values mapped onto observed FVC measurements on a new sample that was not used for training the model. The GCMs showed that the predicted FVC tracked longitudinally, although to a lesser extent than the actual FVC measurements. The test-retest reliability was lower than the actual FVC, but the reliability was still at commensurate with other commonly used outcome measures in ALS.

Using the features and parameters estimated from the final training model, a predicted FVC measure was obtained for each observation in the test set. Prediction accuracy was evaluated using the mean absolute error (MAE) between the observed FVC measures and the FVC measures predicted according to the model:

$$MAE = {\sum }_{i = 1}^{n} {\sum }_{j = 1}^{{t}_{i}} \vert {FVC}_{ij} - {ij}_{} \vert$$

where i is the i-th participant, j is the j-th observation for the i-th participant, FVC is the observed FVC value, and [symbol] is the predicted FVC value. The MAE is interpreted in the same units as the original outcome, which in this case is FVC L. Lower MAE scores indicated better prediction accuracy.

A growth curve model (GCM) was employed to evaluate the longitudinal change. A GCM is a mixed-effects model where the dependent variable is the outcome of interest and the primary predictor is the time variable. The following GCM was used:

FVCᵢⱼ=b₀ᵢ+b₁ᵢ·tᵢⱼ+eᵢⱼ

where FVCᵢⱼ is the FVC value for individual i at time j; b₀ᵢ is the intercept (e.g., the expected FVC measure when tᵢⱼ=0) for participant i, which follows a normal distribution with mean intercept β₀ (fixed effect) and a standard deviation; b₁ᵢ is the mean slope (i.e., rate of change) for all individuals; tᵢⱼ is the value of the time variable (e.g., number of days since enrollment) for individual i at time j; and eᵢⱼ is the residual term for individual i at time j.

Two separate GCMs were made: one for the observed FVC measures and one for the FVC measures predicted by the model. The time variable was the number of days since enrollment in the study. Prediction accuracy of the final model on the training sample was evaluated using leave-one-participant-out cross-validation. All correlations reported were adjusted for the repeated measurements per participant. The repeatability of the MPT prediction was estimated using the intra-class correlation (ICC), the within-person standard error of measurement (SEM), and the within-person coefficient of variation.

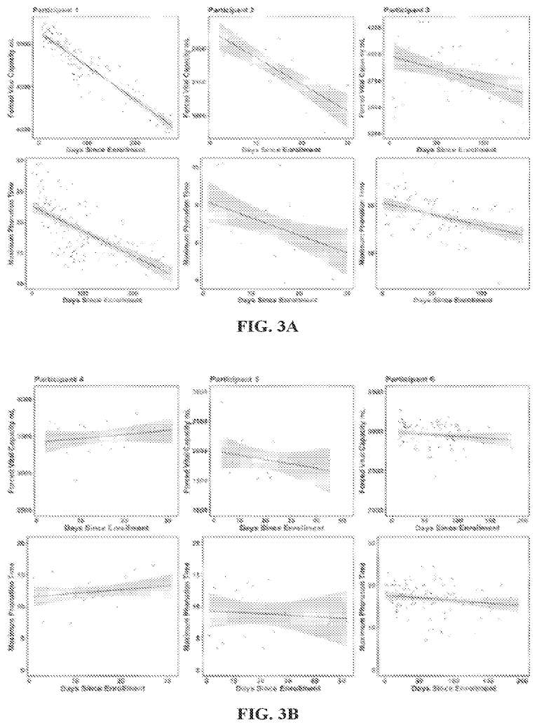

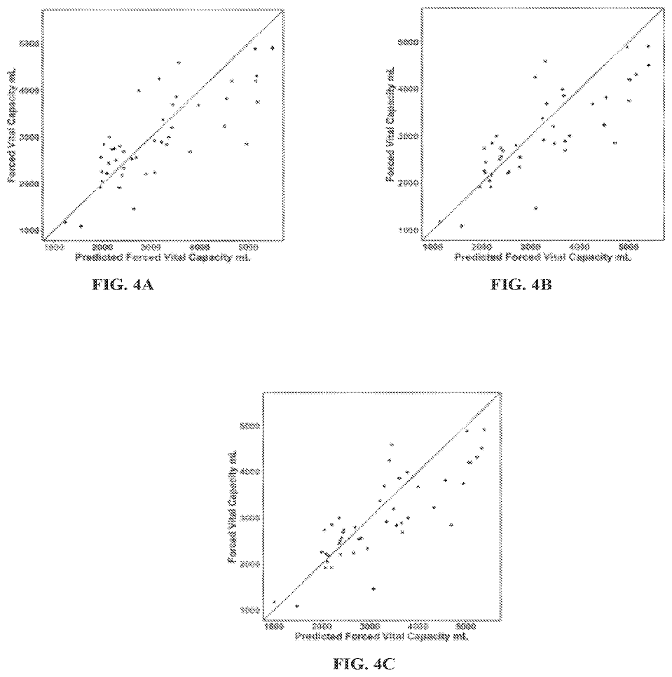

FIG. 3A and FIG. 3B display individual trajectories of participants from the training set for FVC (top row) and MPT (bottom row) with an MAE was 0.47 L (relative MAE=14%) and the correlation coefficient r was 0.72. The model was then trained to obtain FVC predictions on the test sample and evaluated the performance on that sample. In the test sample, the MAE was 0.58 L (relative MAE=19.5%) and the correlation coefficient r was 0.80, meaning that on average, the predicted FVC deviated 0.58 L from the observed FVC in the test sample. Finally, the prediction accuracy was evaluated using the average of 3 and 5 MPT measurements for prediction (i.e., the average of the observation overlapping with FVC, the one or two before, and the one or two after), and found that prediction accuracy increased when more MPT measurements were used. The model fit results are shown in the table below, including the MAE, relative MAE, and r. FIG. 4A shows predicted and observed FVC in a scatterplot (using only the original single overlapping MPT and FVC without any averaging). The diagonal line shows where the points should be if the prediction were perfect, and the distance between each individual point the diagonal line indicates the size of the prediction error.

FIG. 4B and FIG. 4C show the predicted and observed FVC scatterplots when using the average of 3 and 5 MPT measurements.

| Model | MAE (L) | r |
|---|---|---|
| Training set: leave-one-participant-out cross-validation | .47 | .72 |
| Testing set: predict FVC using single observation | .58 | .80 |
| Testing set: predict FVC using the three nearest | .56 | .80 |
| observations | | |
| Testing set: predict FVC using the five nearest | .54 | .80 |
| observations | | |

### Model Fit

A GCM was fitted to the observed and predicted FVC values in the training sample (via cross-validation) and test sample, and was evaluated for the longitudinal slopes (mean rate of change) for both sample sets. The below table shows the GCM parameters for the observed and predicted FVC models using both the cross-validated training data and the test data. The fixed-effects intercepts indicate the expected intercept (expected FVC at the start of the study), the fixed-effects slopes indicate the expected rates of change (expected decline FVC in L per month), the intercepts standard deviation indicates how much participants varied in their unique intercepts (how different participants were at the start of the study), and the residual standard deviation is how much each observation deviated from each participant's unique trajectory. The slopes had fixed effects but not random effects. The final models reported in this paper all converged appropriately. Both GCMs yielded significantly negative slopes, indicating that both observed and predicted FVC were declining throughout the study.

| | | | | Training | Training |
|---|---|---|---|---|---|
| | | Test Data | Test Data | Data | Data |
| | | Model: | Model: | Model: | Model: |
| | | Observed | Predicted | Observed | Predicted |
| Parameter | | FVC | FVC | FVC | FVC |
| Fixed effects | Intercept | 3.28 (.24) | 3.27 (.23) | 3.64 (.18) | 3.61 (.15) |
| | (Liters) | | | | |
| | Slope | −.037 (.01) | −.017 (.004) | −.027 (.005) | −.024 (.006) |
| | (L/month) | | | | |
| Random | Intercepts | 1.15 | 1.13 | 1.14 | .94 |
| effects | standard | | | | |
| | deviation | | | | |
| | Residual | .17 | .33 | .19 | .24 |
| | standard | | | | |
| | deviation | | | | |

### Mean (Standard Error) Parameters for the GCMs

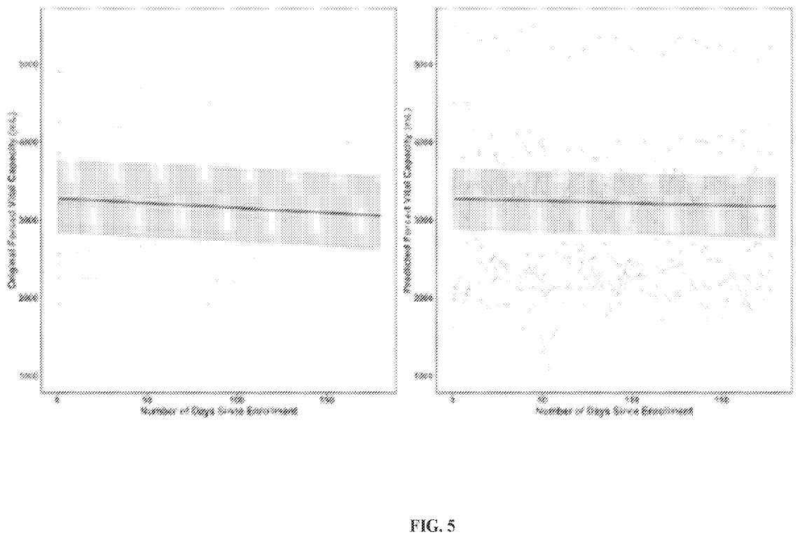

FIG. 5 shows the observed data for the FVC and predicted FVC side-by-side. For clearer visualization of the trajectories, only those participants with at least 15 phonation measurements were included in the plots. However, the full sample was used for the analyses. The dark blue lines show the predicted trajectory according to the GCM and the blue shades show the 95% confidence band, which was estimated using the predicted population interval method. As shown in the plots, both predicted and observed FVC values had very similar intercepts and declining trajectories; however, the predicted FVC values declined at a slower rate than the observed FVC values.

The test-retest repeatability scores were computed for observed FVC and predicted FVC values on the training and test data. The ICC ranges from 0 to 1, where higher scores indicate higher repeatability. SEM is the within-person standard deviation, and it is expressed in the observed units of FVC (L), such that lower values indicate lower variability (and therefore higher repeatability). The CV is the within-person variability (standard deviation) divided by the mean of the data, and it is expressed as a percentage, such that lower values indicate lower variability (and therefore higher repeatability). The below table shows the repeatability scores for the observed and predicted FVC in the training and test sets.

| Model | ICC | SEM | CV |
|---|---|---|---|
| Original FVC in Training Data | 0.97 | 0.19 | 6% |
| Predicted FVC in Training Data | 0.94 | 0.24 | 9% |
| Original FVC in Testing Data | 0.97 | 0.22 | 6% |
| Predicted FVC in Testing Data | 0.92 | 0.34 | 15% |

### Repeatability Scores

### Digital Therapeutics

In some embodiments, the systems, methods, and media disclosed herein provide one or more digital therapeutics for improving pulmonary function or respiratory health or one or more status indicators of a disease or condition (such as the disease areas disclosed in Table 1). In some cases, an electronic device such as a smartphone or tablet is configured with software to provide digital therapeutics, for example, cognitive behavioral therapy. Such digital therapeutics can be stand-alone or integrate the evaluation of pulmonary function as disclosed herein to enable monitoring of disease progression or status and/or therapeutic efficacy.

Various forms of digital therapy are contemplated, including current digital therapeutics already available to the public. For example, such digital therapeutics include a digital therapeutic app that provides treatment management for COPD and asthma in conjunction with a smart sensor attached to the subject's inhaler that tracks usage. As another example, a digital therapeutic app for COPD provides virtual physical therapy through customized daily COPD-relevant exercises. In some embodiments, the pulmonary evaluation software application is separate and distinct from the digital therapeutic. Alternatively, one can be integrated into the other as a single stand-alone software application (e.g., a mobile app on a smartphone, tablet, or other smart device). For example, the pulmonary function evaluation software may be integrated as an API within a digital therapeutic app, including a third party digital therapeutic.

In some embodiments, the systems, methods, and media disclosed herein provide telemedicine to the subject, for example, messaging and/or live phone/video conferencing with a human healthcare provider. In some cases, such as when there is not a human healthcare provider currently available, a digital caregiver/doctor is provided allowing the subject to interact with an automated dialogue system to obtain further information on their needs. This information can be provided to the healthcare provider to respond either in real-time or later such as via messaging.

In some embodiments, the systems, methods, and media disclosed herein provide digital therapeutic monitoring via an iterative loop in which one or more elicitation tasks are prompted to the subject, audio data is collected, the audio data is processed to extract relevant features associated with one or more respiratory measures, the relevant features are analyzed using an algorithm (e.g., trained machine learning model) to generate an evaluation (e.g., forced vital capacity or other respiratory measure), and this process is repeated one or more times while the subject undergoes one or more digital therapies. For example, a baseline evaluation of a particular measure of pulmonary function may be obtained initially prior to treatment, and then periodic evaluations can be obtained remotely via a user device such as a smartphone. This approach for remote evaluation of pulmonary function is convenient and enables more frequent data collection resulting in a more robust data set for treatment evaluation or simply for monitoring of the subject's disease or condition. This remote approach is also helpful for improving clinical trial participation because more people can participate if they do not have to travel on-site for evaluation. Once the baseline evaluation is established, continued periodic evaluations can be used to then inform further treatment. For example, if the respiratory measures do not show improvement in pulmonary function, then the treatment regimen may be modified by changing the digital therapeutic to a different therapeutic, referring to a doctor or specialized for follow-up evaluation (e.g., to determine why there is no improvement or to identify an alternative treatment), or modifying the digital therapeutic (e.g., changing frequency, duration, or other aspects of the therapeutic).

In some embodiments, the systems, methods, and media disclosed herein provide a rewards system for the evaluation and/or digital therapeutic process. Subjects may be hard to motivate to stay on track with a digital therapeutic, and so a rewards system can incentivize continued or more diligent participation. In some cases, the subject is rewarded with a digital token such as points which may be tradeable for digital or real world rewards (e.g., coupons, gift certificates or gift cards, cash, or other suitable rewards). These rewards can be provided through a software application, website, or network portal. In some embodiments, the software application used to provide the digital therapeutic comprises an integrated rewards system for rewarding user compliance with the digital therapeutics and/or evaluation of pulmonary function. The rewards system can provide tracking of evaluations of pulmonary function and/or digital therapeutics (e.g., timeline, frequency, duration, compliance with a preset regimen or schedule), rewards accrued (e.g., points and/or rewards), statistics or metrics on the evaluations themselves (e.g., respiratory measures, changes in disease status as assessed by the respiratory measures as well as subject questionnaires), or any combination thereof.

### Web Applications

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application in some embodiments is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

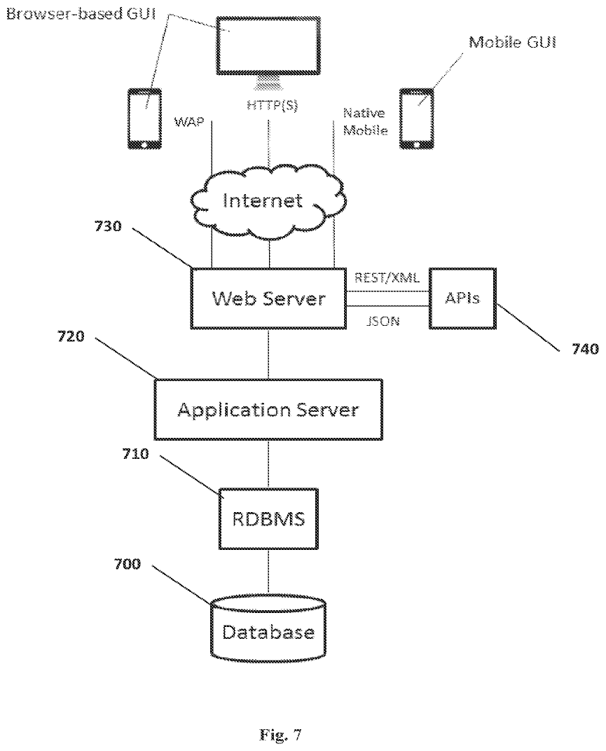

Referring to FIG. 7, in a particular embodiment, an application provision system comprises one or more databases 700 accessed by a relational database management system (RDBMS) 710. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 720 (such as Java servers, .NET servers, PHP servers, and the like) and one or more web servers 730 (such as Apache, IIS, GWS and the like). The web server(s) optionally expose one or more web services via app application programming interfaces (APIs) 740. Via a network, such as the Internet, the system provides browser-based and/or mobile native user interfaces.

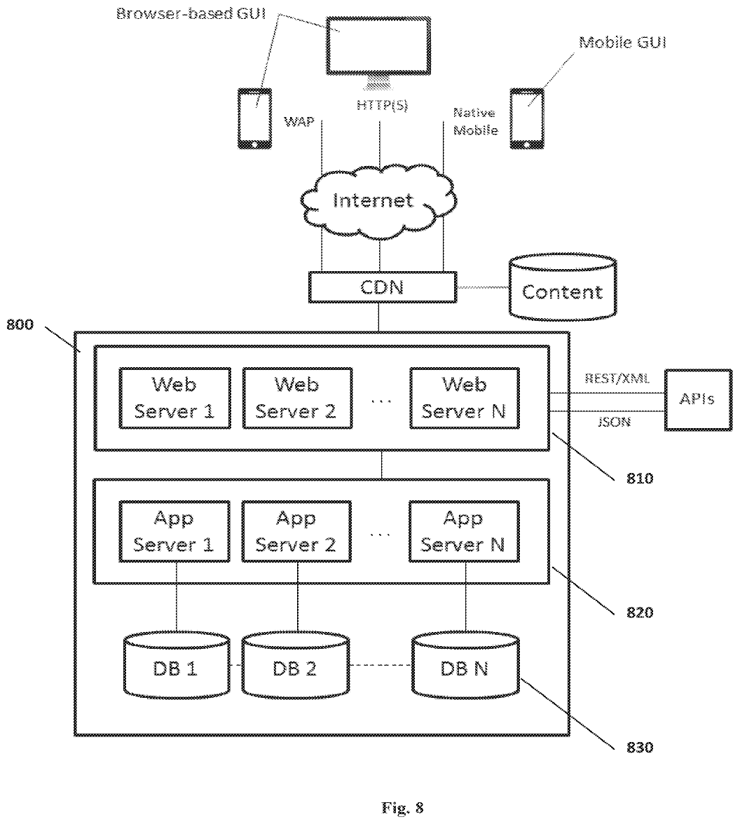

Referring to FIG. 8, in a particular embodiment, an application provision system alternatively has a distributed, cloud-based architecture 800 and comprises elastically load balanced, auto-scaling web server resources 810 and application server resources 820 as well synchronously replicated databases 830.

### Mobile Applications

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Applications

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB.NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Computing Systems

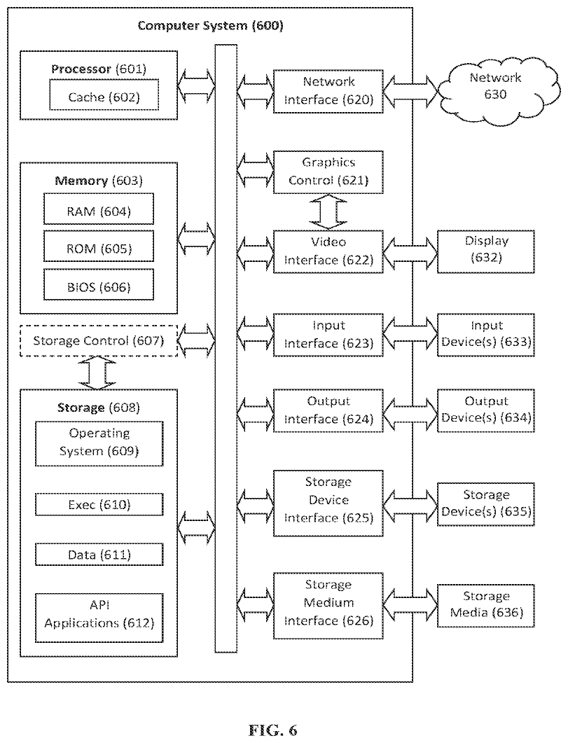

Referring to FIG. 6, a block diagram is shown depicting an exemplary machine that includes a computer system 600 (e.g., a processing or computing system) within which a set of instructions can execute for causing a device to perform or execute any one or more of the aspects and/or methodologies for static code scheduling of the present disclosure. The components in FIG. 6 are examples only and do not limit the scope of use or functionality of any hardware, software, embedded logic component, or a combination of two or more such components implementing particular embodiments.

Computer system 600 may include one or more processors 601, a memory 603, and a storage 608 that communicate with each other, and with other components, via a bus 640. The bus 640 may also link a display 632, one or more input devices 633 (which may, for example, include a keypad, a keyboard, a mouse, a stylus, etc.), one or more output devices 634, one or more storage devices 635, and various tangible storage media 636. All of these elements may interface directly or via one or more interfaces or adaptors to the bus 640. For instance, the various tangible storage media 636 can interface with the bus 640 via storage medium interface 626. Computer system 600 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 600 includes one or more processor(s) 601 e.g., central processing units (CPUs) or general purpose graphics processing units (GPGPUs)) that carry out functions. Processor(s) 601 optionally contains a cache memory unit 602 for temporary local storage of instructions, data, or computer addresses. Processor(s) 601 are configured to assist in execution of computer readable instructions. Computer system 600 may provide functionality for the components depicted in FIG. 6 as a result of the processor(s) 601 executing non-transitory, processor-executable instructions embodied in one or more tangible computer-readable storage media, such as memory 603, storage 608, storage devices 635, and/or storage medium 636. The computer-readable media may store software that implements particular embodiments, and processor(s) 601 may execute the software. Memory 603 may read the software from one or more other computer-readable media (such as mass storage device(s) 635, 636) or from one or more other sources through a suitable interface, such as network interface 620. The software may cause processor(s) 601 to carry out one or more processes or one or more steps of one or more processes described or illustrated herein. Carrying out such processes or steps may include defining data structures stored in memory 603 and modifying the data structures as directed by the software.

The memory 603 may include various components (e.g., machine readable media) including, but not limited to, a random access memory component (e.g., RAM 604) (e.g., static RAM (SRAM), dynamic RAM (DRAM), ferroelectric random access memory (FRAM), phase-change random access memory (PRAM), etc.), a read-only memory component (e.g., ROM 605), and any combinations thereof. ROM 605 may act to communicate data and instructions unidirectionally to processor(s) 601, and RAM 604 may act to communicate data and instructions bidirectionally with processor(s) 601. ROM 605 and RAM 604 may include any suitable tangible computer-readable media described below. In one example, a basic input/output system 606 (BIOS), including basic routines that help to transfer information between elements within computer system 600, such as during start-up, may be stored in the memory 603.

Fixed storage 608 is connected bidirectionally to processor(s) 601, optionally through storage control unit 607. Fixed storage 608 provides additional data storage capacity and may also include any suitable tangible computer-readable media described herein. Storage 608 in some embodiments is used to store operating system 609, executable(s) 610, data 611, applications 612 (application programs), and the like. Storage 608 can also include an optical disk drive, a solid-state memory device (e.g., flash-based systems), or a combination of any of the above. Information in storage 608 may, in appropriate cases, be incorporated as virtual memory in memory 603.

In one example, storage device(s) 635 are removably interfaced with computer system 600 (e.g., via an external port connector (not shown)) via a storage device interface 625. Particularly, storage device(s) 635 and an associated machine-readable medium may provide non-volatile and/or volatile storage of machine-readable instructions, data structures, program modules, and/or other data for the computer system 600. In one example, software may reside, completely or partially, within a machine-readable medium on storage device(s) 635. In another example, software may reside, completely or partially, within processor(s) 601.

Bus 640 connects a wide variety of subsystems. Herein, reference to a bus may encompass one or more digital signal lines serving a common function, where appropriate. Bus 640 in some embodiments is any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 600 may also include an input device 633. In one example, a user of computer system 600 may enter commands and/or other information into computer system 600 via input device(s) 633. Examples of an input device(s) 633 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 633 in some embodiments are interfaced to bus 640 via any of a variety of input interfaces 623 (e.g., input interface 623) including, but not limited to, serial, parallel, game port, USB, FIREWIRE, THUNDERBOLT, or any combination of the above.

In particular embodiments, when computer system 600 is connected to network 630, computer system 600 may communicate with other devices, specifically mobile devices and enterprise systems, distributed computing systems, cloud storage systems, cloud computing systems, and the like, connected to network 630. Communications to and from computer system 600 in some embodiments are sent through network interface 620. For example, network interface 620 may receive incoming communications (such as requests or responses from other devices) in the form of one or more packets (such as Internet Protocol (IP) packets) from network 630, and computer system 600 may store the incoming communications in memory 603 for processing. Computer system 600 may similarly store outgoing communications (such as requests or responses to other devices) in the form of one or more packets in memory 603 and communicated to network 630 from network interface 620. Processor(s) 601 may access these communication packets stored in memory 603 for processing.

Examples of the network interface 620 include, but are not limited to, a network interface card, a modem, and any combination thereof. Examples of a network 630 or network segment 630 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 630, may employ a wired and/or a wireless mode of communication. In general, any network topology may be used.

Information and data can be displayed through a display 632. Examples of a display 632 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 632 can interface to the processor(s) 601, memory 603, and fixed storage 608, as well as other devices, such as input device(s) 633, via the bus 640. The display 632 is linked to the bus 640 via a video interface 622, and transport of data between the display 632 and the bus 640 can be controlled via the graphics control 621. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 632, computer system 600 may include one or more other peripheral output devices 634 including, but not limited to, an audio speaker, a printer, a storage device, and any combinations thereof. Such peripheral output devices in some embodiments are connected to the bus 640 via an output interface 624. Examples of an output interface 624 include, but are not limited to, a serial port, a parallel connection, a USB port, a FIREWIRE port, a THUNDERBOLT port, and any combinations thereof.

In addition or as an alternative, computer system 600 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein in some embodiments are implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein in some embodiments are implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor in some embodiments is a microprocessor, but in the alternative, the processor in some embodiments is any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein in some embodiments are embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium is integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions in some embodiments are implemented as program modules, such as functions, objects, Application Programming Interfaces (APIs), computing data structures, and the like, that perform particular tasks or implement particular abstract data types. In light of the disclosure provided herein, those of skill in the art will recognize that a computer program in some embodiments is written in various versions of various languages.

The functionality of the computer readable instructions in some embodiments are combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Databases

In some embodiments, the platforms, systems, media, and methods disclosed herein include one or more databases, or use of the same. In view of the disclosure provided herein, those of skill in the art will recognize that many databases are suitable for storage and retrieval of audio, spirometric, medical, and health information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

### Machine Learning

In some embodiments, machine learning algorithms are utilized to determine a forced vital capacity (FVC) based on speech acoustics or speech data. In some embodiments, machine learning algorithms are utilized to determine a respiratory illness based on the FVC data. In some embodiments, machine learning algorithms are utilized to determines a maximum phonation time (MPT) based on a collected ambient noise sample and a sustained phonation sample.

In some embodiments, the machine learning algorithms herein employ one or more forms of labels including but not limited to human annotated labels and semi-supervised labels. In some embodiments, the machine learning algorithm utilizes regression modeling, wherein relationships between predictor variables and dependent variables are determined and weighted. In one embodiment, for example, the FVC is a dependent variable and is derived from the MPT data, the audio data, or both. In another embodiment, for example, the probability that the accessory dwelling unit can be constructed on the property is a dependent variable and is derived from the potential accessory dwelling unit landmark and the plurality of property structure indicators.

The human annotated labels can be provided by a hand-crafted heuristic. For example, the hand-crafted heuristic can comprise examining differences between public and county records. The semi-supervised labels can be determined using a clustering technique to find properties similar to those flagged by previous human annotated labels and previous semi-supervised labels. The semi-supervised labels can employ a XGBoost, a neural network, or both.

In some embodiments, the potential accessory dwelling unit landmark on the property is detected using a distant supervision method. In some embodiments, the probability that the accessory dwelling unit can be constructed on the property is determined using a distant supervision method. The distant supervision method can create a large training set seeded by a small hand-annotated training set. The distant supervision method can comprise positive-unlabeled learning with the training set as the ‘positive’ class. The distant supervision method can employ a logistic regression model, a recurrent neural network, or both. The recurrent neural network can be advantageous for Natural Language Processing (NLP) machine learning.

Examples of machine learning algorithms can include a support vector machine (SVM), a naïve Bayes classification, a random forest, a neural network, deep learning, or other supervised learning algorithm or unsupervised learning algorithm for classification and regression. The machine learning algorithms can be trained using one or more training datasets.

In some embodiments, a machine learning algorithm is used to select catalogue images and recommend project scope. A non-limiting example of a multi-variate linear regression model algorithm is seen below: probability=A₀+A₁(X₁)+A₂(X₂)+A₃(X₃)+A₄(X₄)+A₅(X₅)+A₆(X₆)+A₇(X₇) . . . wherein (A₁, A₂, A₃, A₄, A₅, A₆, A₇, . . . ) are “weights” or coefficients found during the regression modeling; and X, (X₁, X₂, X₃, X₄, X₅, X₆, X₇, . . . ) are data collected from the User. Any number of Aᵢ and Xᵢ variable can be included in the model. For example, in a non-limiting example wherein there are 7 Xᵢ terms, X₁ is the number of property record depictions, X₂ is the number of potential accessory dwelling unit landmarks, and X₃ is the probability that the accessory dwelling unit can be constructed on the property. In some embodiments, the programming language “R” is used to run the model.

In some embodiments, the first machine learning algorithm determines a potential accessory dwelling unit landmark on the property based on a plurality of aerial images.

In some embodiments, the first machine learning algorithm is trained by a neural network comprising: a first training module creating a first training set comprising a set of aerial images predetermined as having a potential accessory dwelling unit landmark and a set of aerial images predetermined as not having a potential accessory dwelling unit landmark; and a first training module training the neural network using the first training set; a second training module creating a second training set for second stage training comprising the first training set and the aerial images incorrectly detected as having a potential accessory dwelling unit landmark after the first stage of training; and training the neural network using the second training set.

In some embodiments, the second machine learning algorithm determines the probability that the accessory dwelling unit can be constructed on the property based on the potential accessory dwelling unit landmark and the plurality of property structure indicators. Alternatively, in some embodiments, training the first machine learning algorithm comprises multiple steps. In a first step, an initial model is constructed by assigning probability weights to predictor variables. In a second step, the initial model is used to “recommend” property structure indicators. In a third step, the validation module accepts verified data regarding the property structure indicators and feeds back the verified data. At least one of the first step, the second step, and the third step can repeat one or more times continuously or at set intervals.

In some embodiments, the second machine learning algorithm is trained by: constructing an initial model by assigning probability weights to predictor variables based on the potential accessory dwelling unit landmark and the property structure indicators; and adjusting the probability weights based on the verified data.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

What is claimed is:

1. A system comprising:
a memory to store instructions;
a processor configured to execute the instructions stored in the memory;
wherein the system is specially configured to execute the instructions stored in the memory via the processor to cause the system to evaluate pulmonary function, by performing operations including:
(a) receiving audio data of a subject;
(b) extracting one or more acoustic features from the audio data;
(c) analyzing the one or more acoustic features using a predictive algorithm to generate an evaluation of pulmonary function for the subject, wherein the subject is undergoing a clinical trial that comprises the evaluation for pulmonary function to assess status of a disease area associated with pulmonary function; and
wherein the subject participates in the clinical trial at via prompting by the system requesting the subject to provide the audio data as input into the system remotely through an electronic device comprising a microphone.

2-34. (canceled)

35. The system of claim 1, wherein the disease area is a respiratory condition or disease affecting bloody oxygenation or homeostasis or a neurodegenerative disease affecting pulmonary function.

36. The system of claim 1, wherein the disease area comprises one of:
ALS, COPD, asthma, cystic fibrosis, COVID-19; and
wherein the evaluation of pulmonary function comprises one or more of: a predicted forced vital capacity, forced expiratory volume, peak expiratory flow, mid-expiratory flow rate, forced inspiratory vital capacity, forced expiratory time, respiration rate, respiration rhythm, respiration quality, pause rate, cough events, maximum phonation time, vocal quality, hypernasality, or any combination thereof.

37. The system of claim 1, wherein the audio data is collected via the microphone without requiring a spirometer via operations including:
providing instructions to the subject to perform a vocal task;
collecting the audio data during performance of the vocal task by the subject at the microphone; and
processing the audio data at the electronic device locally where the audio data was captured by the microphone or alternatively transmitting the audio data from the electronic device to the system for remote processing of the audio data.

38. The system of claim 1:
wherein the predictive algorithm comprises a trained machine learning model having been trained on the one or more acoustic features selected from the group comprising: MPT, measures of pitch, loudness, or vocal quality;
wherein the trained machine learning model is configured for analyzing one or more demographics features in new audio data captured from the subject which forms no part of training data for the trained machine learning model; and
wherein the one or more demographics features are provided to the trained machine learning model comprise one or more of: age, height, gender, and weight.

39. The system of claim 1, further comprising:
monitoring the pulmonary function of the subject over time based on generating the evaluation of pulmonary function over time; and
providing a software application accessible through a user electronic device to the subject for monitoring the pulmonary function of the subject over time.

40. The system of claim 39, wherein the software application is configured to prompt the subject with one or more elicitation tasks and record one or more audio recordings of the subject to obtain the audio data.

41. The system of claim 40, wherein the one or more acoustic features comprises one or more features recited in Table 2.

42. The system of claim 1:
wherein the audio data is received from an electronic device used by the subject to collect the audio data remotely;
wherein the electronic device is not a specialized device for measuring respiratory function; and
wherein the electronic device is a portable user electronic device selected from the group comprising: a mobile phone, a smartphone, a tablet, a desktop computer, or a laptop computer.

43. The system of claim 1, further comprising:
transmitting from the system to the electronic device having the microphone, an installable software application accessible to, or downloadable by, the electronic device responsive to a request from the electronic device; and
wherein the installable software application configures the electronic device to remotely collect the audio data on behalf of the system via the microphone and to transmit the audio data collected back to the system.

44. The system of claim 43, wherein the installable software application is a mobile application specially configured to provide clinical trial registration.

45. The system of claim 1:
wherein the subject is undergoing one or more digital therapeutics for improving or treating pulmonary function or mitigating or slowing progression of decline of pulmonary function of the subject; and
wherein the system is specially configured to execute the instructions to perform operations further comprising: generating as output specific to the subject, one or more digital therapeutics recommended for improving or treating pulmonary function or mitigating or slowing progression of decline of pulmonary function of the subject.

46. The system of claim 45:
wherein the one or more digital therapeutics comprises one or more physical therapy exercises for improving pulmonary function.

47. The system of claim 45:
wherein the one or more digital therapeutics are provided through an integrated software application that provides the one or more digital therapeutics in combination with the evaluation of pulmonary function.

48. The system of claim 45, further comprising:
operating a rewards system for incentivizing the subject into compliance with the evaluation of pulmonary function, or the one or more digital therapeutics, or both.

49. The system of claim 1, wherein the predictive algorithm generates evaluations of pulmonary function with a correlation coefficient of at least 0.65, 0.70, 0.75, or 0.80 when comparing predicted evaluations to ground truth evaluations for at least 100 independent samples.

50. A computer-implemented method performed by a system having at least a processor and a memory therein to execute instructions for evaluating pulmonary function, wherein the method comprises:
(a) receiving audio data of a subject;
(b) extracting one or more acoustic features from the audio data;
(c) analyzing the one or more acoustic features using a predictive algorithm to generate an evaluation of pulmonary function for the subject, wherein the subject is undergoing a clinical trial that comprises the evaluation for pulmonary function to assess status of a disease area associated with pulmonary function; and
wherein the subject participates in the clinical trial at via prompting by the system requesting the subject to provide the audio data as input into the system remotely through an electronic device comprising a microphone.

51. The computer-implemented method of claim 50:
wherein the disease area is a respiratory condition or disease affecting bloody oxygenation or homeostasis or a neurodegenerative disease affecting pulmonary function;
wherein the disease area comprises one of: ALS, COPD, asthma, cystic fibrosis, COVID-19; and
wherein the evaluation of pulmonary function comprises one or more of: a predicted forced vital capacity, forced expiratory volume, peak expiratory flow, mid-expiratory flow rate, forced inspiratory vital capacity, forced expiratory time, respiration rate, respiration rhythm, respiration quality, pause rate, cough events, maximum phonation time, vocal quality, hypernasality, or any combination thereof.

52. Non-transitory computer readable storage media having instructions stored thereupon that, when executed by a system having at least a processor and a memory therein, the instructions cause the processor to execute instructions for evaluating pulmonary function, by performing the following operations:
(a) receiving audio data of a subject;
(b) extracting one or more acoustic features from the audio data;
(c) analyzing the one or more acoustic features using a predictive algorithm to generate an evaluation of pulmonary function for the subject, wherein the subject is undergoing a clinical trial that comprises the evaluation for pulmonary function to assess status of a disease area associated with pulmonary function; and
wherein the subject participates in the clinical trial at via prompting by the system requesting the subject to provide the audio data as input into the system remotely through an electronic device comprising a microphone.

53. The non-transitory computer readable storage media of claim 52:
wherein the disease area is a respiratory condition or disease affecting bloody oxygenation or homeostasis or a neurodegenerative disease affecting pulmonary function;
wherein the disease area comprises one of: ALS, COPD, asthma, cystic fibrosis, COVID-19; and
wherein the evaluation of pulmonary function comprises one or more of: a predicted forced vital capacity, forced expiratory volume, peak expiratory flow, mid-expiratory flow rate, forced inspiratory vital capacity, forced expiratory time, respiration rate, respiration rhythm, respiration quality, pause rate, cough events, maximum phonation time, vocal quality, hypernasality, or any combination thereof.
